(19) United States
(12) Patent Application Publication
(10) Pub. No.: US 2010/0069776 A1
(43) Pub. Date: Mar. 18, 2010

(54) DIAGNOSING AND MONITORING NEUROLOGICAL PATHOLOGIES AND STATES

(76) Inventors: Bradley Greger, Centerville, UT (US); Paul House, Salt Lake City, UT (US); Kyle Thomson, Salt Lake City, UT (US)

Correspondence Address: MCDERMOTT WILL & EMERY LLP, 18191 VON KARMAN AVE., SUITE 500, IRVINE, CA, 92612-7108, US

(21) Appl. No.: 12/554,839

(22) Filed: Sep. 4, 2009

Related U.S. Application Data
(60) Provisional application No. 61/094,393, filed on Sep. 4, 2008.

(51) Int. Cl. A61B 5/0478

(52) U.S. Cl. 600/544

(57) ABSTRACT

Certain embodiments of the present invention provide for the detection and monitoring of multiple micro-scale neurological signals indicative of neurological state, neurological activity, and/or neuropathology. By examining such micro-scale neurological signals, a care provider may make more accurate differential diagnoses, identify the most efficacious treatment strategy, and/or track the efficacy of treatment. In some embodiments, analysis of micro-scale electrophysiological signals can be used in the diagnosis, treatment decisions, and monitoring of several neurological disorders, e.g. epilepsy, movement disorders, and psychiatric disorders. In some embodiments, different cortical areas can be mapped, for example, to define boundaries between healthy and/or pathological neural tissue.

### RELATED APPLICATION

The present application claims the benefit of priority under 35 U.S.C. §119 from U.S. Provisional Patent Application Ser. No. 61/094,393, entitled “METHOD AND APPARATUS FOR DIAGNOSING AND MONITORING NEUROLOGICAL PATHOLOGIES,” filed on Sep. 4, 2008, which is hereby incorporated by reference in its entirety for all purposes.

### FIELD OF THE INVENTION

The present invention relates to methods diagnosing and monitoring neurological pathologies and states.

### BACKGROUND OF THE INVENTION

Prior art methods and devices for detecting and/or monitoring neurological activity rely primarily on analysis of electroencephalography (EEG) and electrocorticography (ECog) signals. EEG and ECog signals are recorded at relatively low sampling rates from macroscopic areas of the brain, and do not provide high resolution spatial and temporal information of neurological state, neurological activity, and/or neuropathology.

### SUMMARY OF THE INVENTION

Certain embodiments of the present invention provide for the detection and monitoring of multiple micro-scale neurological signals indicative of neurological state, neurological activity, and/or neuropathology. By examining such micro-scale neurological signals, a physician, psychiatrist, clinician, or other care provider may make more accurate differential diagnoses, identify the most efficacious treatment strategy, and/or track the efficacy of treatment.

In some embodiments, analysis of micro-scale electrophysiological signals can be used in the diagnosis, treatment decisions, and monitoring of several neurological disorders, e.g. epilepsy, movement disorders, and psychiatric disorders.

In some embodiments, different cortical areas can be mapped, for example, to define boundaries between healthy and/or pathological neural tissue.

In certain embodiments, a method, of localizing a neurological pathology within the brain, comprises at each one of a plurality of electrode arrays located at or near the surface of a brain, detecting electrical activity occurring within the brain, wherein each of the electrode arrays comprises a plurality of electrodes, for each one of the plurality of electrode arrays, determining a direction of propagation of the electrical activity based on the electrical activity detected by the electrode array, and determining a location of the neurological pathology based on the determined directions of propagation of the electrical activity.

In certain embodiments, the electrical activity comprises inter-itcal spikes and the neurological pathology comprises an epileptic focus.

In certain embodiments, the detecting the electrical activity occurring within the brain at each of the plurality of electrode arrays comprises detecting an electrical signal at each of the electrodes of the electrode array, and the determining the direction of propagation of the electrical activity at each one of the plurality of electrode arrays comprises determining a gradient of electrical signals across a plane of the electrode array based on the electrical signals detected by the electrodes of the electrode array, and determining the direction of propagation of the electrical activity based on the determined gradient.

In certain embodiments, the electrical signals comprise electrical potentials.

In certain embodiments, the detecting the electrical activity occurring within the brain at each of the plurality of electrode arrays comprises detecting an electrical signal at each one of the plurality of electrodes of the electrode array, and the determining the direction of propagation of the electrical activity at each one of the plurality of electrode arrays comprises determining a wave front of the electrical activity across the electrode array based on the electrical signals detected by the electrodes of the electrode array, and determining the direction of propagation of the electrical activity based on the determined wave front.

In certain embodiments, the determining the wave front of the electrical activity across the electrode array comprises determining at which electrodes of the electrode array the respective electrical signal is above a threshold and at which electrodes of the electrode array the respective electrical signal is below the threshold, forming a boundary between the electrodes corresponding to electrical signals above the threshold and the electrodes corresponding to electrical signals below the threshold, and determining the wave front of the electrical activity based on the boundary.

In certain embodiments, a method, of characterizing neurological activity, comprising over a time period, detecting a local field potential occurring in a group of neurons, during the time period, detecting action potentials within the group of neurons, determining a value of a feature indicative of a relationship between the detected local field potential and the detected action potentials, and outputting to an output device information indicative of the value of the feature.

In certain embodiments, if the value of the feature is different from a normal or standard range of values, the method outputs to the output device information indicative of the difference.

In certain embodiments, the feature comprises a temporal correlation between the detected local field potential and the detected action potentials.

In certain embodiments, the local field potential comprises an interictal spike.

In certain embodiments, the value of the feature comprises one or more of an action potential firing rate and a number of action potentials during the detected local field potential.

In certain embodiments, the feature comprises a temporal correlation between a phase of the detected local field potential and the detected action potentials.

In certain embodiments, the feature comprises a decrease in action potential firing rate during a time period following the local field potential.

In certain embodiments, the feature comprises a frequency of the local field potential.

In certain embodiments, the method further comprises outputting to the output device information indicative of a neurological state based on the difference.

In certain embodiments, the method further comprises outputting to the output device a differential diagnosis based on the difference.

In certain embodiments, a method, of mapping cortical areas in a brain using an electrode array comprising a plurality of electrodes, comprises at each electrode of the electrode array located at or near the surface of a brain, detecting an electrical signal from the brain, correlating the electrical signals between different pairs of the electrodes of the electrode array to generate a correlation map of the electrode array, and determining a boundary between a first cortical area and a second cortical area of the brain based on the correlation map.

In certain embodiments, the determining the boundary between the two cortical areas comprises identifying a first area of the correlation map indicating a higher level of correlation than a second area of the correlation map, and mapping the first and second areas of the correlation map onto the first and second cortical areas of the brain, respectively.

In certain embodiments, the electrical signals are detected at two or more of before, during and after performance of a function by the subject of the brain.

In certain embodiments, the function comprises one or more of the following: a motor movement, a calculation, a memory recall, counting, sleep, awake and a thought task.

In certain embodiments, functional mapping of first and second functional areas of the brain is performed based on the boundary

In certain embodiments, a method, of localizing brain electrical activity, comprises at each of a plurality of electrode arrays located at or near a surface of a brain, detecting electrical activity occurring within the brain, wherein each of the electrode arrays comprises a plurality of electrodes, for each of the electrode arrays, determining a first function, describing a relationship of measured values of an electrical parameter at each of the electrodes within that array, determining a second function, describing a relationship among the first functions of the plurality of the electrode arrays, and based on the second function, determining a location of the electrical activity within the brain.

In certain embodiments, the electrical parameter comprises voltage.

In certain embodiments, the electrical parameter comprises current.

### BRIEF DESCRIPTION OF THE DRAWINGS

FIG. 1A shows a perspective view of a microelectrode array according to an embodiment of the present invention.

FIG. 1B shows a top view of a microelectrode array according to an embodiment of the present invention.

FIG. 1C shows pictures of a prior art, standard ECog array; a prior art, micro-ECog array; and a microelectrode array according to an embodiment of the present invention.

FIG. 1D shows a picture of a prior art, standard ECog; a prior art array, micro-ECog array; and a microelectrode array according to an embodiment of the present invention implanted in a patient.

FIG. 1E shows a block diagram of a system for recording and analyzing data from the microelectrode array according to an embodiment of the present invention.

FIG. 2 shows electrical traces from a prior art, standard ECog array and electrical races from a microelectrode array according to an embodiment of the present invention.

FIG. 3 shows correlation analysis maps of each electrode in a micro-ECog array.

FIG. 4 shows correlation analysis maps of each electrode in a micro-ECog array.

FIG. 5 shows correlation analysis map of each electrode in a microelectrode array according to the present invention.

FIG. 6A shows correlation versus distance for a ECog array.

FIG. 6B shows correlation versus distance for a micro-ECog array.

FIG. 6C shows a correlation versus distance for a microelectrode array according to an embodiment of the present invention

FIG. 7A shows measurement of baseline (awake) neural activity of a cat.

FIG. 7B shows measurement of anesthetized (asleep) neural activity of a cat.

FIGS. 8A and 8B each show a plot of an interictal spike and APs recorded on a single electrode and a spectrogram according to an embodiment of the present invention.

FIG. 9A shows a mean waveform of interictal spikes according to an embodiment of the present invention.

FIG. 9B shows an average spectrogram above a frequency of 300 Hz corresponding to the mean interictal spike in FIG. 9A.

FIG. 9C shows a plot of mean power prior to and after the mean interictal spike in FIG. 9A.

FIG. 10A shows a mean waveform of interictal spikes according to an embodiment of the present invention.

FIG. 10B shows a graph of detected APs during the interictal spikes in FIG. 10A.

FIG. 10C shows a plot of AP firing rate recorded at two neurons.

FIG. 11 shows a waveform of an interictal spike according to an embodiment of the present invention.

FIGS. 12A-12Fp each show a plot of voltage levels across the electrodes of a microelectrode array at different times during the interictal spike in FIG. 11 and derivatives of the gradient of the across the plane of the microelectrode array.

FIGS. 13A and 13B histograms indicating the directionality of multiple interictal spikes for two patients according to an embodiment of the present invention.

FIGS. 14A and 14B illustrate a method for determining the direction of an interictal spike based on a time of arrival of a wave front of the interictal spike across a microelectrode array according to an embodiment of the present invention.

FIG. 15 illustrates a method for determining the location of an epileptic focus based on the directions of interictal spikes determined at different locations of the brain according to an embodiment of the present invention.

FIGS. 16A and 16B shows correlation analysis maps for an array implanted in the epidural space in a nonhuman primate and an array implanted in the subdural space in a human patient, respectively, according to an embodiment of the present invention.

FIG. 17 shows a boundary between two cortical areas determined based on the correlation analysis map in FIG. 16B.

### DETAILED DESCRIPTION OF THE INVENTION

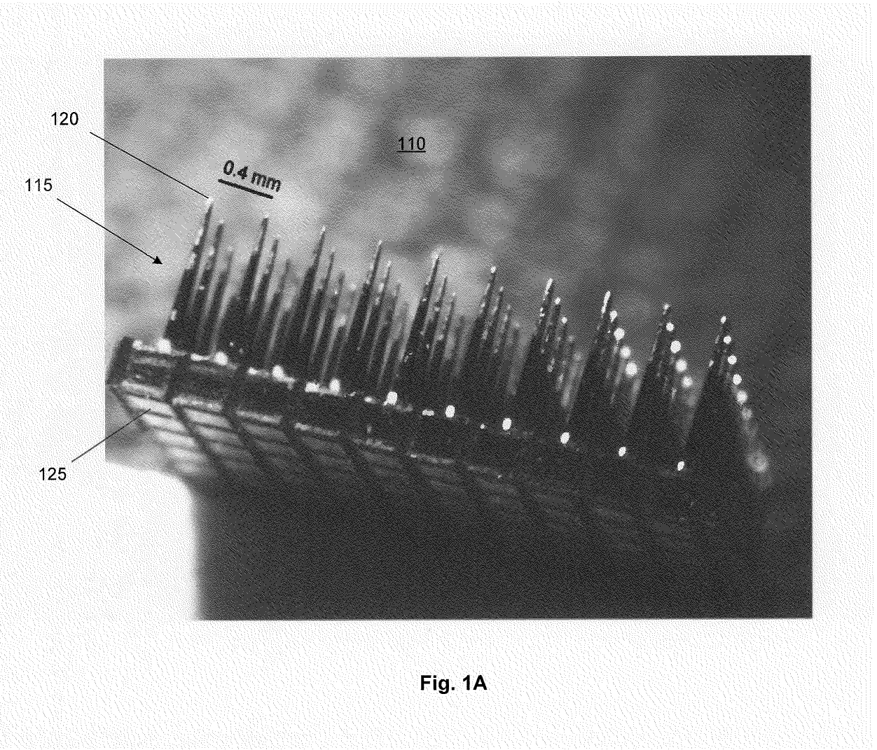

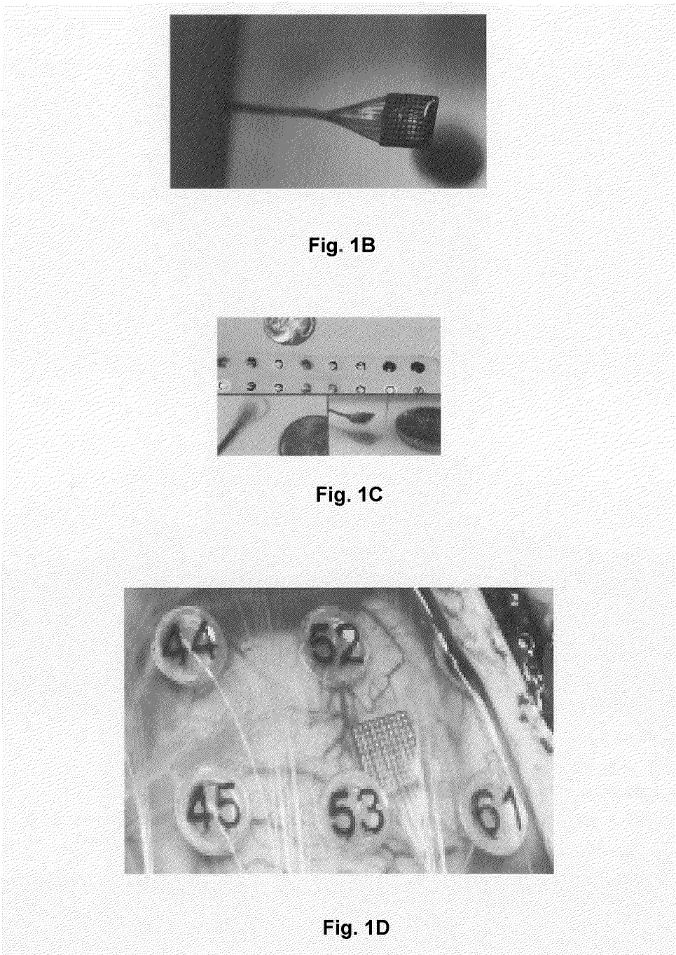

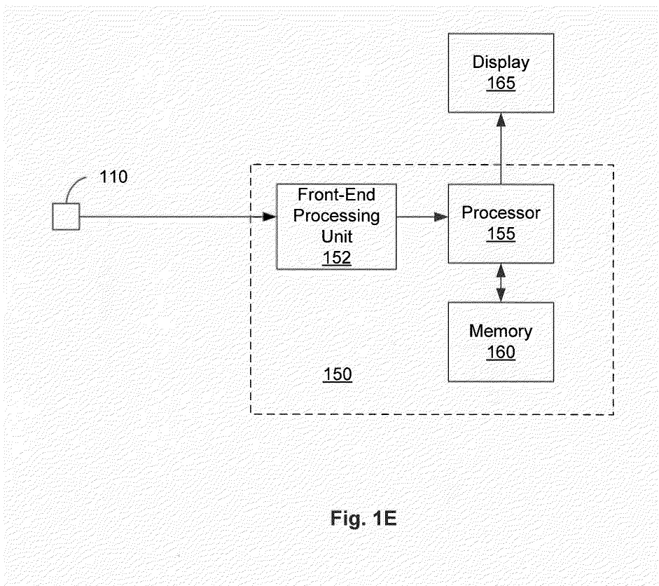

FIG. 1A shows a perspective view of a microelectrode array 110 according to an embodiment of the present invention. The microelectrode array 110 comprises a plurality of electrodes 115 (e.g., 100) arranged in an array (e.g., 10×10). In one embodiment, the microelectrode array 110 is capable of measuring electrical activity over a large surface area of the brain (e.g., cortex), while each electrode 115 of the array is capable of measuring electrical activity in a localized area of a brain.

In the example shown in FIG. 1A, the microelectrode array is capable of providing a multi-channel interface to the cerebral cortex. The microelectrode array 110 has a large number of 1.0-1.5 mm long electrodes 115 (typically 100 in a 10×10 square grid) that project out from a very thin (0.2 mm) substrate. The electrodes 115 are separated from each other by 0.4 mm. The large number of penetrating electrodes 115 of the microelectrode array 110 presents a very large surface area to the cortex allowing the implanted microelectrode 110 array to “self anchor” to cortical tissues. Such an array has an advantage in that it “integrates” with the cortical tissues and, therefore, “floats” with respect to the cranium. As the cortex moves due to respiration and blood pumping, or to body motions, the array moves with the cortex, thereby producing little or no relative motion between the neurons and the array's active electrode tips 120. This design feature therefore, produces an extremely stable interface with the surrounding neurons.

Each electrode 115 may be about 80 microns in diameter at its base and taper to a sharpened tip 120 that has a radius of curvature of two to three microns. In this embodiment, the electrodes resist bending during the insertion process and displace about 4% of the cortical volume in which they are inserted.

In one embodiment, each electrode 115 may be electrically isolated from its neighboring electrodes 115 with a moat of glass that surrounds each electrode's base. The electrodes 115 may comprise doped silicon with tips that are metalized with iridium oxide to facilitate electronic to ionic transduction. Conduction of electrical signals along the length of each electrode 115 is achieved by the doped silicon used in its fabrication. Electrode impedances (measured with a 100 nanoamp, 1 kHz sine wave current) may range from a few tens to a few hundreds of k Ωs. The electrodes 115, with the exception of the iridium oxide coated tips 120, may be insulated with a 2 micron thick coat of parylene or other insulator.

Each electrode 115 may have a bonding pad 125 on the back on the rear of the substrate. In this embodiment, an electrical connection may be made to each electrode 115 by wire bonding an insulated 25 micron diameter wire to the corresponding bond pad, and connecting the wire to a percutaneous connector. The bond pads 115 and the lead wires may be potted to provide electrical insulation and mechanical strength, as shown in FIG. 1B.

FIG. 1E is block diagram showing an example of a system 150 for recording and processing electrical signals from the microelectrode array 110. The system 150 includes a front-end processing unit 152, a processor 155, and a memory 160. The front-end processing unit 152 conditions the electrical signals from the microelectrode array 110 for processing by the processor. The front-end processing unit 152 may include one or more of the following components: amplifiers (e.g., low-noise amplifiers) for amplifying the electrical signals, a filter for isolating electrical signals within a desired frequency bandwidth, and an analog-to-digital converter for digitizing the electrical signals for processing by the processor. Some or all of the above components may also be implanted in the patient with the microelectrode array 110.

The processor 155 may comprise a general purpose processor, a digital signal processors (DSPs), application specific integrated circuit (ASICs), discrete hardware components, or any combination thereof. Methods for processing and analyzing the electrical signals from the array 110 according to various embodiments of the invention discussed below may be embodied in software code that is stored in the memory 160 and executed by the processor 155. The memory 160 may comprise any computer-readable media known in the art including volatile memory, nonvolatile memory, a Random Access Memory (RAM), a flash memory, a Read Only Memory (ROM), a removable disk, a CD-ROM, a DVD, any other suitable storage device, or a combination thereof.

The processor 155 may store raw electrical signals, processed electrical signals, and/or results of analysis performed on electrical signals in the memory. The raw electrical signals may refer to the digitized electrical signals from the front-end processing unit 152 and processed electrical signals may refer to electrical signals further processed by the processor 155. For example, the processor 155 may digitally filter the electrical signals to remove certain frequency components of the electrical signals. The processor 155 may also output raw electrical signals, processed electrical signals, and/or results of analysis to an output device, including, but not limited to, a display 165 for viewing by a neurologist, a printer for generating a computer readout, a computer-readable media, and/or to another computer via a computer network connection.

Recordings of action potentials (APs) and local field potentials (LFPs) from the cerebral cortexes of several human patients and experimental animals were performed. The APs may be repetitive waveforms which are distinguishable for individual neurons and a LFP may be a sum of electrical potential from a group of neurons. Patients and/or subjects were implanted with up to three devices: a standard ECog grid (silver wires and disk electrodes of FIG. 1D), a micro-ECog grid (green wire of FIG. 1D), and a microelectrode array according to an embodiment of the present invention (gold wires of FIG. 1D). High quality micro-ECog signals, local field potentials (LFPs), and action potentials (APs) were recorded from the implanted devices. Using such micro-scale signals (micro-ECog, LFPS, APs) clinically significant phenomenon where detected, such as fundamental changes in the information processing state of the brain during different levels of consciousness and a directional propagation of seizure related neurological activity.

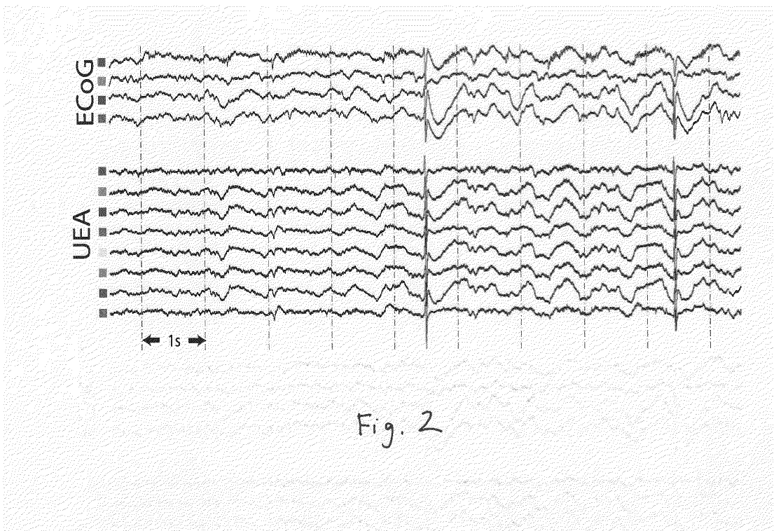

FIG. 2 shows raw traces of 10 seconds of data. The top four traces are data recorded from a ECoG array and the lower eight traces are time-aligned data recorded from a microelectrode array according to an embodiment of the present invention. This shows that the epileptic activity in the form of interictal spikes is seen on both the large ECoG array, as well as microelectrode array. The microelectrode array provides a higher spatial resolution, as well as micro-scale activity associated with the known epileptiform activity seen on both devices. This activity can also be observed on microECoG devices.

A patient undergoing localization of epileptic focus was implanted with a micro-ECog; a standard ECog; and a microelectrode array according to an embodiment of the present invention. Correlation analysis was run on epochs of 5 minutes of data using the equations:

$$\begin{matrix}C ( m , p ) = E [ ( m - {μ}_{m} ) ( p - {μ}_{p} ) ] ∀ m , p & ( 1 ) \\ R ( m , p ) = \frac{C ( m , p )}{\sqrt{C ( m , m ) C ( n , p )}} & ( 2 )\end{matrix}$$

in which Equation (1) calculates a covariance matrix C where m and p are the data from channels [1, 2, . . . N], where N is the number of electrodes in the array, and μₘ and μₚ are the respective means of these channels. A correlation matrix R was determined from the normalized covariance matrix, as shown in Equation (2).

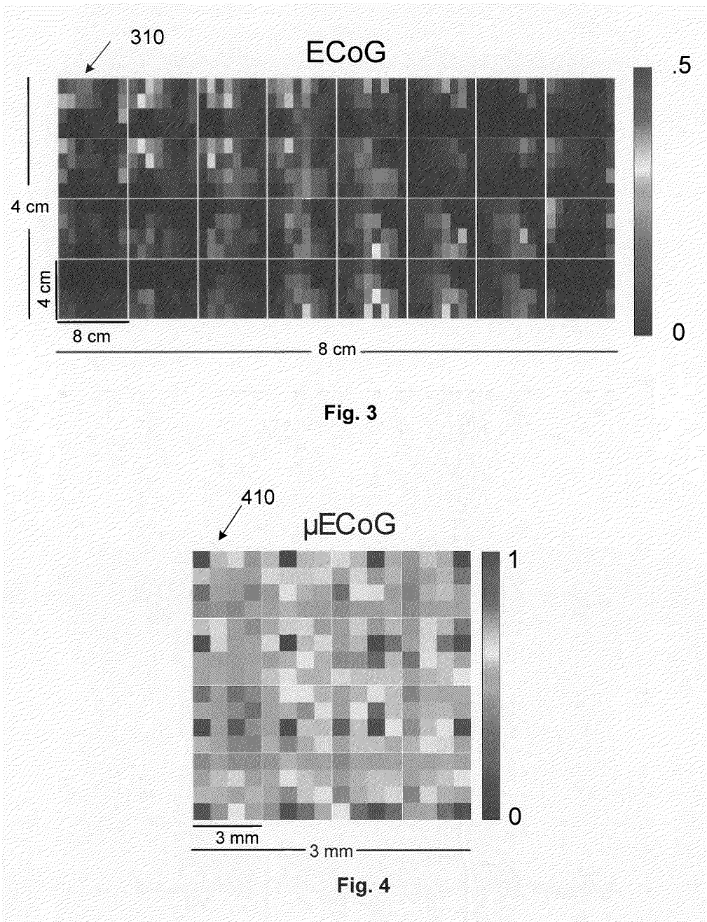

FIG. 3 shows an ECog correlation analysis map, in which each square 310 shows an electrode's correlation to all other electrodes in the ECog array. The squares 310 match the layout of the electrodes in the ECog array so that a square 310 at a given column and row in FIG. 3 shows the correlation of the electrode at the given column and row in the ECog array to all other electrodes in the ECog array. For example, the upper-left square 310 in FIG. 3 shows the correlation of the upper-left electrode in the ECog array to all other electrodes in the ECog array. The square 310 at each location of the array comprises a miniature replica of the array indicating the pairwise correlation of the electrode at that location to all other electrodes in the array. The miniature replica of the array in each square matches the layout of the electrodes in the ECog array. Thus, for a square 310 at a given location in the array, a miniature square at a given column and row in the miniature replica of the square 310 shows the correlation of the electrode at the location of the square 310 to the electrode at the given column and row in the array. In the example in FIG. 3, the 32 squares match the layout of an ECog array, in which the electrode spacing was 1 centimeter.

FIG. 4 shows a micro-ECog correlation analysis map, in which each square 410 shows an electrode's correlation to all other electrodes. The squares 410 match the layout of the electrodes in the micro-ECog array so that a square 410 at a given column and row in FIG. 4 shows the correlation of the electrode at the given column and row in the micro-ECog array to all other electrodes in the micro-ECog array. The square 410 at each location of the array comprises a miniature replica of the array indicating the pairwise correlation of the electrode at that location to all other electrodes in the array, as explained above. The 16 squares also match the layout of the micro-ECog, in which electrode size was 40 microns with 1 mm spacing between electrodes. In FIG. 4, local field potentials were recorded from the surface of the cortex using non-penetrating electrodes.

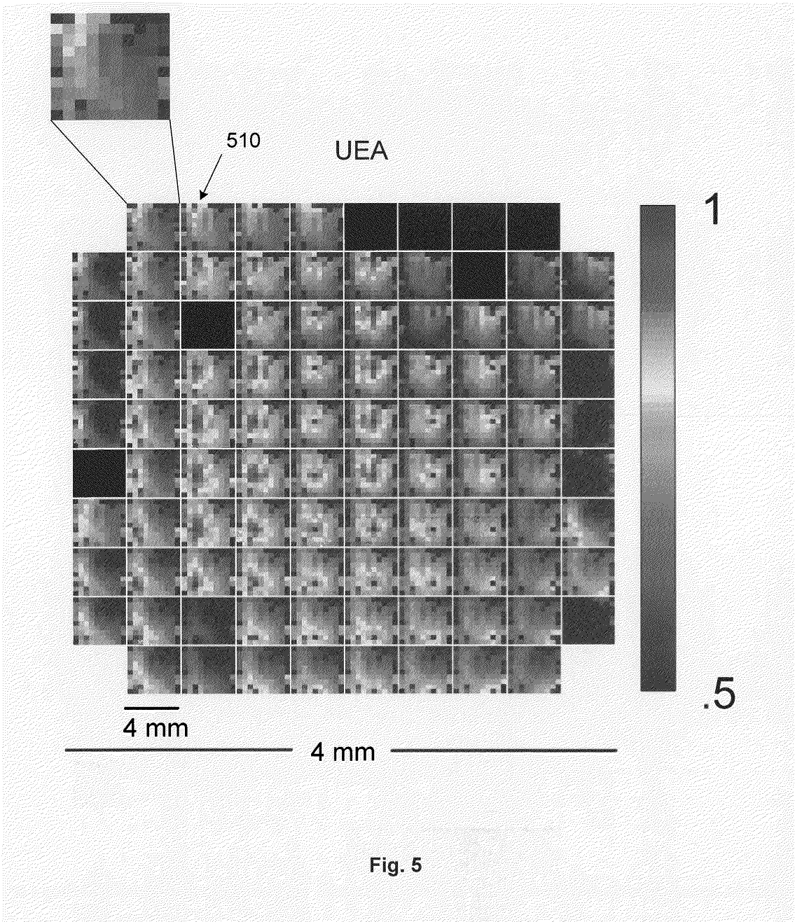

FIG. 5 shows an array correlation analysis map for the microelectrode array according to an embodiment of the present invention. Each square 510 shows an electrode's correlation to all other electrodes. The squares 510 match the layout of the electrodes in the microelectrode array so that a square 510 at a given column and row in FIG. 5 shows the correlation of the electrode at the given column and row in the array to all other electrodes in the array. The square 510 at each location of the array comprises a miniature replica of the array indicating the pairwise correlation of the electrode at that location to all other electrodes in the array, as explained above. FIG. 5 shows an enlarged view of one of the squares 510. In the example shown in FIG. 5, the electrode size was 40 μm and the electrode spacing was 400 μm.

In FIG. 5, local field potentials were recorded from micro-electrodes that penetrated into the cerebral cortex.

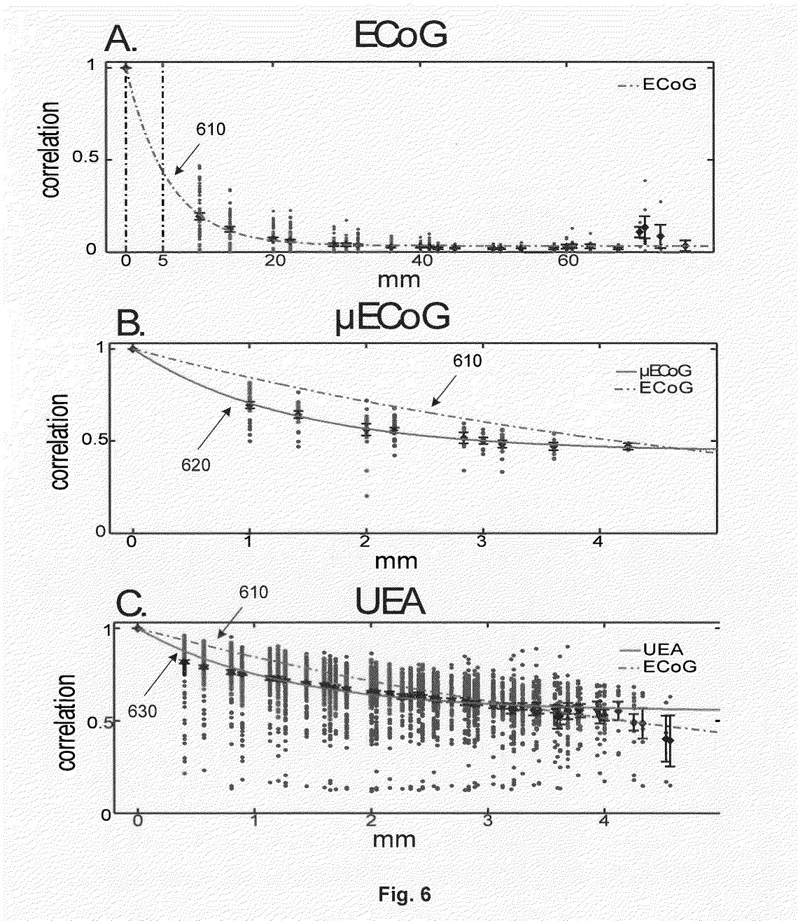

FIG. 6A shows a plot of correlation versus distance for the ECog array. Each point in the plot represents the correlation between a pair of electrodes in the ECog array that are spaced apart by the corresponding distance in the plot. In FIG. 6A, a curve 610 is fitted to the points in the plot using a non-linear fitting technique (e.g., second-order polynomial). The curve 610 represents the drop off in correlation versus distance for the ECog array.

FIG. 6B shows a plot of correlation versus distance for the micro-ECog array. Each point in the plot represents the correlation between a pair of electrodes in the micro-ECog array that are spaced apart by the corresponding distance in the plot. In FIG. 6A, a curve 620 is fitted to the points in the plot using a non-linear fitting technique. The curve 620 represents the drop off in correlation versus distance for the micro ECog array. For comparison, the curve 610 for the ECog array is shown also FIG. 6B. FIG. 6B was generated from micro-scale signals recorded on non-penetrating micro-electrodes.

FIG. 6C shows a plot of correlation versus distance for the microelectrode array according an embodiment of the present invention. Each point in the plot represents the correlation between a pair of electrodes in the microelectrode array that are spaced apart by the corresponding distance in the plot. Different points corresponding to the same distance in the plot represent different combinations of electrode pairs in the microelectrode array that are spaced apart by the same distance. The plot is FIG. 6C contains a much larger number of points than the plots in FIG. 6A and 6B because the microelectrode array comprises more electrodes than the ECog and micro-ECog arrays. FIG. 6C was generated from micro-scale signals recorded on penetrating micro-electrodes.

In FIG. 6C, a curve 630 is fitted to the points in the plot using a non-linear fitting technique (e.g., second order polynomial). The curve 630 represents the drop off in correlation versus distance for the microelectrode array according to an embodiment of the present invention. For comparison, the curve 610 for the ECog array is shown FIG. 6C. As shown in FIG. 6C, the curves 610 and 630 show a much steeper drop in correlation versus distance for the microelectrode array compared to the ECog array. The steeper drop in correlation versus distance indicates that the channels in the microelectrode array are less correlated with one another than the channels in the ECog array, where each channel corresponds to electrical signals from a particular electrode. As a result, the microelectrode array has greater channel independence per unit area and therefore greater spatial resolution than the ECog array. Thus, the microelectrode array according to an embodiment of the present invention is capable of resolving electrical activity in very small area of the cortex, such as electrical activity of individual neurons. The higher spatial resolution allows the microelectrode array to measure micro-scale signals, such as APs from individual neurons.

Measurements of neurological activity of awake and anesthetized cats were taken. There was a substantial change in the frequency of the LFPs across the levels of anesthesia. It was observed that LFPs were tightly correlated with the occurrence of APs, so that the change in LFP frequency resulted in a concomitant change in AP firing rate and synchrony. Since many neural pathologies occur together with changes in the state of neurological information processing, the relationship between APs and LFPs can serve as an indicator for different neurological pathologies, and therefore aid in diagnosis and determination of appropriate treatment. This indicator can also be used to track the efficacy of treatment over time, for monitoring the state of neural information processing, etc.

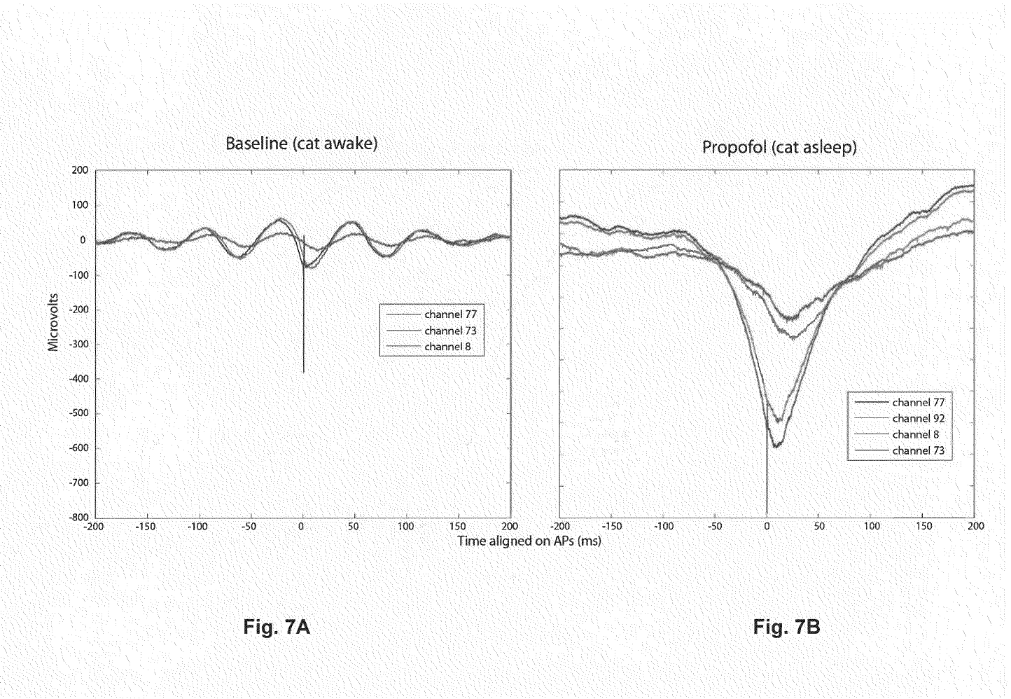

FIGS. 7A and 7B show the LFP (sinusoids) aligned on the occurrence of individual APs (vertical line at time zero) at two different levels of consciousness, awake and asleep. FIG. 7A shows the relatively higher frequency LFPs surrounding the APs when the subject is awake and capable of processing neural information. FIG. 7A shows the relatively lower frequency LFPs surrounding the APs when the subject in deeply anesthetized and incapable of processing neural information. It is the LFP and AP signals, and the relationship between these two micro-scale signals, which can serve as a useful indicator of neurological state or level of anesthesia. In FIGS. 7A and 7B the APs appear as vertical lines at time zero.

In both FIGS. 7A and 7B, the APs occur at the negative going phases of the LFPs. Thus, the phase relationship between LFPs and the AP firing remain approximately the same when the subject is awake and asleep. When the subject is asleep, the LFPs have a lower frequency causing the negative going phases of the LFPs to occur less frequently. As a result, there are fewer opportunities for AP firings, which are correlated with the negative going phases of the LFPs. Thus, changes in the LFP frequency result in a concomitant change in AP firing rate and synchrony.

In the example in FIG. 7A, the APs were measured at the electrode on channel 77 of the array and LFPs were measured at the electrodes on channels 8, 73, and 77 of the array. The LFPs measured on channels 73 and 77 show a strong relationship with the APs measured on channel 77 due to their close spatial proximity. In the example in FIG. 7B, the APs were measured at the electrode on channel 77 of the array and LFPs were measured at the electrodes on channels 8, 73, 77, and 92 of the array.

The relationship between LFPs and APs can serve as an indicator for different neurological pathologies and/or neurological states, and therefore aid in diagnosis and determination of appropriate treatment. For example, an observed relationship between LFPs and APs recorded with the microelectrode array can be compared to a relationship between LFPs and APs associated with a particular neurological pathology and/or neurological state to determine whether the neurological pathology and/or neurological state is present. Examples of relationships between interictal spikes (type of LFPs) and APs associated with an epileptic pathology are provided below. Further, the efficacy of a treatment for a neurological pathology can be monitored by looking for changes in the relationship between LFPs and APs associated with the neurological pathology.

In one embodiment, the system may record LFPs and APs from a patient's brain using a microelectrode array implanted in the patient and determine a value of a feature that is indicative of the relationship between the LFPs and APs. For example, the feature may comprise a temporal correlation between LFPs and a burst of APs and the value may comprise AP firing rate, number of detected APs, and/or intensity of APs during LFPs. In this example, a value of the feature above a normal range of APs may be indicative of a neurological pathology and/or a neurological state.

In another example, the feature may comprise a temporal correlation between a phase (e.g., negative phase) of LFPs and APs and the value may comprise AP firing rate, number of detected APs, and/or intensity of APs during a phase of the LFPs. In this example, a value of the feature above a normal range of APs may be indicative of a neurological pathology and/or a neurological state.

In another example, the feature may comprise a frequency of oscillation of the LFPs and a temporal correlation of LFPs and APs. In this example, the value of the feature may comprise a frequency of oscillation of the LFPs, which may be indicative of the neural processing state of the brain with a lower frequency being indicative of an unconsciousness state.

When the value of the feature is different from the normal range of values, the system may output information indicative of the difference to an output device, including a display, a printer, a computer-readable media and/or another computer via a compute network. For example, if the difference is indicative of a neurological pathology and/or neurological state, then the system may output a differential diagnosis for the neurological pathology and/or an indicator of the neurological state (e.g., neural processing state of the brain).

The relationships between LFPs and APs for different neurological pathologies may be determined based on relationships recorded from known cases of the neurological pathologies. The neurological pathologies may include, but or not limited to, Amyotroplic Lateral Sclerosis (Lou Gehrig's disease), Huntington's disease, Parkinson's disease, alzheimer's, multi-infarct dementia, and neurological pathologies related to stroke, primary tumor, metastatic tumor, vascular malformation, etc. The relationship between LFPs and APs for a particular neurological pathology my be recorded from a patient known to be inflicted with the neurological pathology or later determined to be inflicted with the neurological pathology (e.g., by an autopsy).

To determine whether a particular neurological pathology is present in a patient, the system may record LFPs and APs from the patient's brain using a microelectrode array and determine a value of a feature indicative of a relationship between the LFPs and APs associated with the neurological pathological. The relationship associated with the neurological pathology may be determined as discussed above. For example, the relationship may be a temporal correlation between a phase (e.g., negative phase) of LFPs and APs, in which the neurological pathology is associated with bursts of APs temporally aligned with the phase of the LFPs. The system may then determine whether a value of the feature is different from a standard or normal range of values. For example, the value of the feature may be an AP firing rate during the phase of the LPFs, in which AP firing rates above a normal range of values are indicative of AP bursts associated with the neurological pathology. The normal range of values may be a range of values lying outside a range of values associated with the neurological pathology. The range of values associated with the neurological pathology may be based on a range of values found in known cases of the neurological pathology. If the value is different from the normal range than the system may determine that the neurological pathology is present or at least not rule out the neurological pathology as a possibility and output an corresponding diagnosis to the output device.

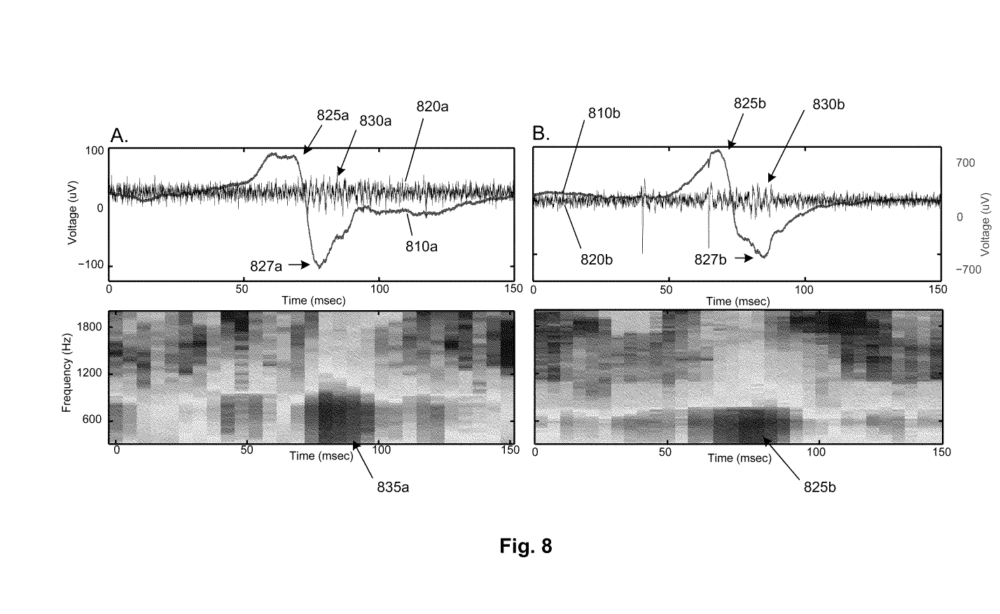

FIG. 8A shows a plot of two electrical traces 810a and 820a recorded from a single electrode in the microelectrode array. Both electrical traces 810a and 820 are derived from the same electrical signal, with electrical trace 810a showing the raw, unfiltered electrical signal and electrical trace 820a showing the electrical signal after being high-pass filtered at 300 Hz. The voltage scale of −700 to 700 μV corresponds to electrical trace 810a and the voltage scale of −100 to 100 μV corresponds to electrical trace 820a.

The electrical trace 810a shows an interictal spike 825a, which is a type of epileptiform having a large distinct waveform. The interictal spike 825a is an epileptic discharge between seizures that propagates through the neural tissue. As shown in FIG. 8A, the interictal spike 825a has a low frequency and can therefore be removed from the electrical signal by high-pass filtering at 300 Hz. The removal of the interictal spike 825a allows the electrical trace 820a to show micro-scale electrical activity in a localized area of the cortex, including Action Potentials (APs) and High Frequency Oscillations (HFOs). An AP may be from an individual neuron caused by an electrical discharge of the neuron. A burst of variable shaped APs may sometimes be referred to as HFOs or fast ripples.

The electrical trace 820a shows a burst of variable shaped APs 830a in a localized area of the cortex when the interictal spike 825a passes the localized area of the cortex. Thus, the bust of APs 830a is temporally correlated with the interictal spike 825a. The burst of APs may originate from an individual neuron, and therefore represent a micro-scale or cellular-level phenomena in the cortex that is temporally correlated with a macro-scale manifestation (interictal spike 825a) of an epileptic pathology. The burst of APs correlated with the epilepsy suggests a dysfunction in specific class of neurotransmitter receptors.

In FIG. 8A, the burst of APs 830 occurs on a negative going phase 827a of the interictal spike 825a. Thus, FIG. 8A demonstrates a correlation between the burst of APs 830a and the phase of the interictal spike 825a, which in this example is the negative going phase 827a of the interictal spike 825a.

FIG. 8A also shows a spectrogram from a frequency of 300 Hz to 2 KHz across 96 channels of the microelectrode array for the interictal spike 825a. The spectrogram shows power above a frequency of 300 Hz, which is above the frequency of the interictal spike 825a. As a result, the spectrogram shows the power of APs. In FIG. 8A, the spectrogram shows a large increase in power 835a above 300 Hz, which is temporally correlated with the interictal spike 825a. The large increase in power above 300 Hz corresponds to the burst of APs, and therefore demonstrates the temporal correlation between the interictal spike 825a and the burst of APs.

FIG. 8B shows similar data and analysis as FIG. 8A and also demonstrates a temporal correlation between an interictal spike 825b and a burst of APs 830b.

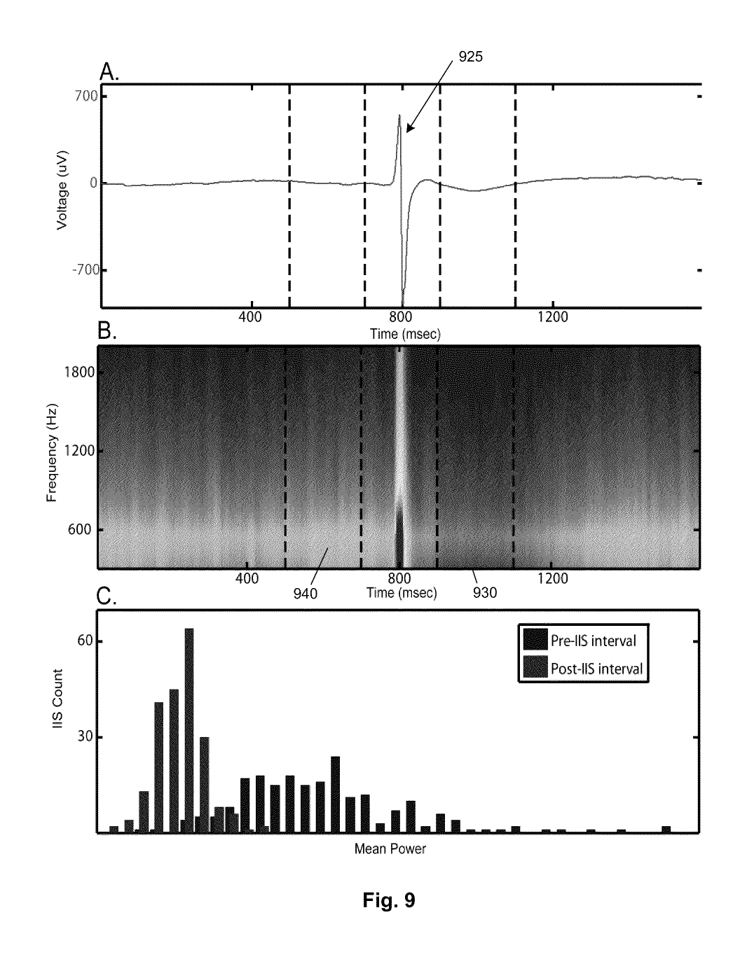

FIG. 9A shows a mean waveform of 216 interictal spikes within the frequency range 0 to 300 Hz. FIG. 9B shows an average spectrogram of power within the frequency range of 300 Hz and 2 KHz, which is aligned with the mean interictal spike 925 in FIG. 9A. The spectrogram shows a large increase in power above 300 Hz, which is temporality correlated with the mean interictal spike 925. The large increase in power above 300 Hz corresponds to an AP burst and therefore demonstrates a temporal correlation between an interictal spike and known epileptic phenomenon.

The spectrogram in FIG. 9B also shows a significant drop in power above 300 Hz in a time period 930 immediately following the mean interictal spike 925 compared to a time period 940 preceding the inter-itcal spike 925. Thus, the spectrogram demonstrates a significant drop in the AP firing rate immediately following an interictal spike 925 compared to the time period prior to the interictal spike 925.

FIG. 9C is a graph showing mean power above 300 Hz from each of the 216 interictal spikes (IIS) binned into pre-IIS (500 to 700 ms) and post-IIS (900 to 1100 ms) groups. The graph in FIG. 9C shows a significant drop in power above 300 Hz in the time period (900 to 110 ms) immediately following an interictal spike compared to the time period preceding the interictal spike (500 to 700 ms).

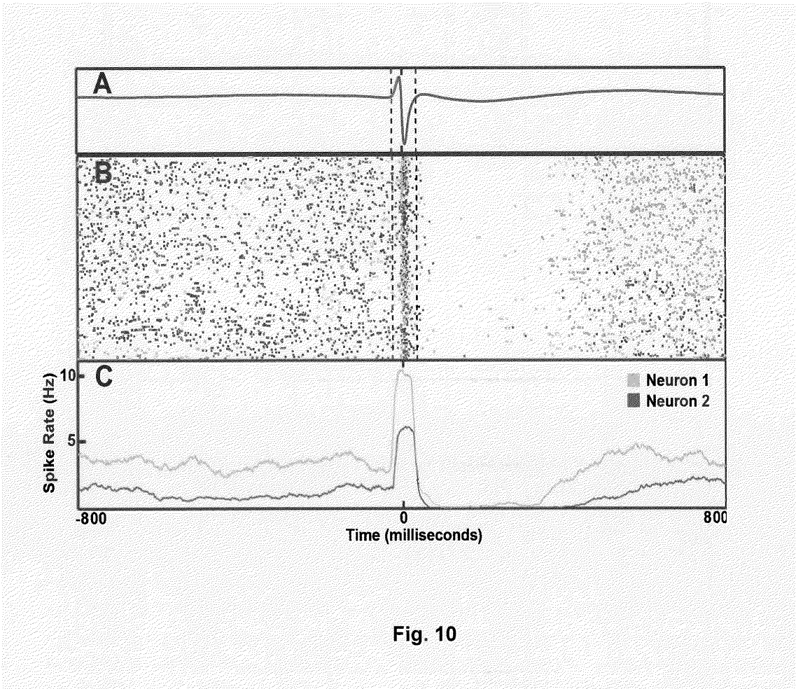

FIG. 10A shows a mean waveform of 658 interictal spikes recorded with a single electrode. FIG. 10B is a graph showing detected APs aligned with the mean interictal spike in FIG. 10A, in which each horizontal line of dots represents detected APs for one of the interictal spikes. APs from two cells were isolated on the electrode and are indicated by two different colors in FIG. 10B.

The APs may be detected by high-pass filtering electrical signals to remove interictal spikes and detecting the high-pass filtered electrical signals above a threshold voltage. In one embodiment, an algorithm may be used to analyze the waveform shapes of the electrical signals above the threshold to further distinguish which of the electrical signals above the threshold are actually APs. For example, the APs may be identified using an algorithm described in Shy Shoham, Matthew R. Fellows, and Richard A. Norman, “Robust Automatic Spike Sorting Using Mixtures of Multivariate t-Distributions,” Journal of Neuroscience Methods, 127(2), 11-122 (2003).

FIG. 10B shows clusters of detected APs temporally aligned with the mean interictal spike, demonstrating a strong temporal correlation between an interictal spike and a burst of APs. FIG. 10B also shows a significant drop in APs in the time period following the mean interictal spike compared to the time period preceding the mean interictal spike.

FIG. 10C shows the mean firing rate of the APs for each of two neurons. FIG. 10C shows an increase in the mean AP firing rate during the mean interictal spike. FIG. 10C also shows an inhibition in the mean AP firing rate immediately following the mean interictal spike compared to the time period preceding the mean interictal spike.

Thus, the data and analysis in FIGS. 8A-10C demonstrate several relationships between interictal spikes and APs. More particularly, the data and analysis in FIGS. 8A-10C demonstrate a temporal correlation between an interictal spike and a burst of APs and a correlation between the phase of the interictal spike and the burst of APs, in which the negative going phase of the interictal spike is temporally aligned with the burst of APs. The data and analysis in FIGS. 9A-10C also demonstrate a suppression in the AP firing rate immediately following an interictal spike compared to a time period prior to the interictal spike. One or more of the above relationships can be used to provide electrical signatures associated with epileptic pathologies.

In one embodiment, electrical signals from a microelectrode array implanted in a patient can be analyzed to detect electrical signatures associated with epileptic pathologies or other neurological pathologies. This may be done, for example, to determine the presence of epilepsy or other neurological pathology, distinguish or categorize sub-types of neurological pathologies and/or indicate an appropriate therapeutic approach or treatment. For example, the system in FIG. 1E or other system may determine the presence of epilepsy by detecting a burst of APs temporally aligned with an interictal spike, detecting a burst of APs temporally aligned with the negative going phase of an inter-itcal spike and/or detecting a drop in AP firing rate immediately following an interictal spike compared to a time period preceding the interictal spike.

The system may detect APs using the techniques discussed above (e.g., high-pass filtering and thresholding) or other techniques. For example, the system may measure mean power above a predetermined frequency threshold (e.g., 300 Hz) and detect a burst of APs based on a detected increase in the mean power above the frequency threshold. The processor may isolate electrical signals above a certain frequency (e.g., 300 Hz) by digitally high-pass filtering electrical signals from the front-end processing unit. Alternatively or in addition, the front-end processing unit may isolate electrical signals above a certain frequency (e.g., 300 Hz) with one or more high-pass filters. The system may measure the AP firing rate based on a number of detected APs within a short time period or other techniques.

In one embodiment, the system may record an interictal spike and APs from a patient's brain using a microelectrode array and determine a value of a feature that is indicative of the relationship between the interictal spike and APs. For example, the feature may comprise a temporal correlation between an inter-itcal spike and a burst of APs and the value may comprise AP firing rate, number of detected APs, and/or intensity of APs during an interictal spike. In this example, a value of the feature that is above a normal range of APs may be indicative of an epileptic pathology or other neurological pathology.

In another example, the feature may comprise a temporal correlation between a phase (e.g., negative phase) of an interictal spike and a burst of APs and the value may comprise AP firing rate, number of detected APs, and/or intensity of APs during a phase of an interictal spike. In this example, a value of the feature above a normal range of APs may be indicative of an epileptic pathology or other neurological pathology.

In another example, the feature may comprise a decrease in AP firing rate immediately following an interictal spike and the value may comprise AP firing rate, number of detected APs and/or intensity of APs in a time period following the interictal spike. In this example, a value of the feature below a normal range of AP firing rate may be indicative of an inhibition in the AP firing rate following the interictal spike, which may be associated an epileptic pathology or other neurological pathology. The normal range of AP firing rate may be based on a range of AP firing rate during a time period prior to the interictal spike.

When the value of the feature is different from the normal range of values, the system may output information indicative of the difference to an output device, including a display, a printer, a computer-readable media and/or another computer via a compute network. For example, if the difference is indicative of an epileptic pathology or other neurological pathology, then the system may output a differential diagnosis for the epileptic pathology or other neurological pathology.

The electrical signals from the microelectrode array may also be analyzed to monitor the progress of epilepsy or other neurological pathology and/or monitor the efficacy of a treatment for the epilepsy or other neurological pathology. For example, the efficacy of a pharmaceutical agent for epilepsy may be monitored by observing whether one or more of the relationships between interictal spikes and APs associated with epilepsy changes as a result of the pharmaceutical agent. For example, a decrease in the AP firing rate during interictal spikes may indicate that the pharmaceutical agent is effective at treating the epilepsy at a cellular-level.

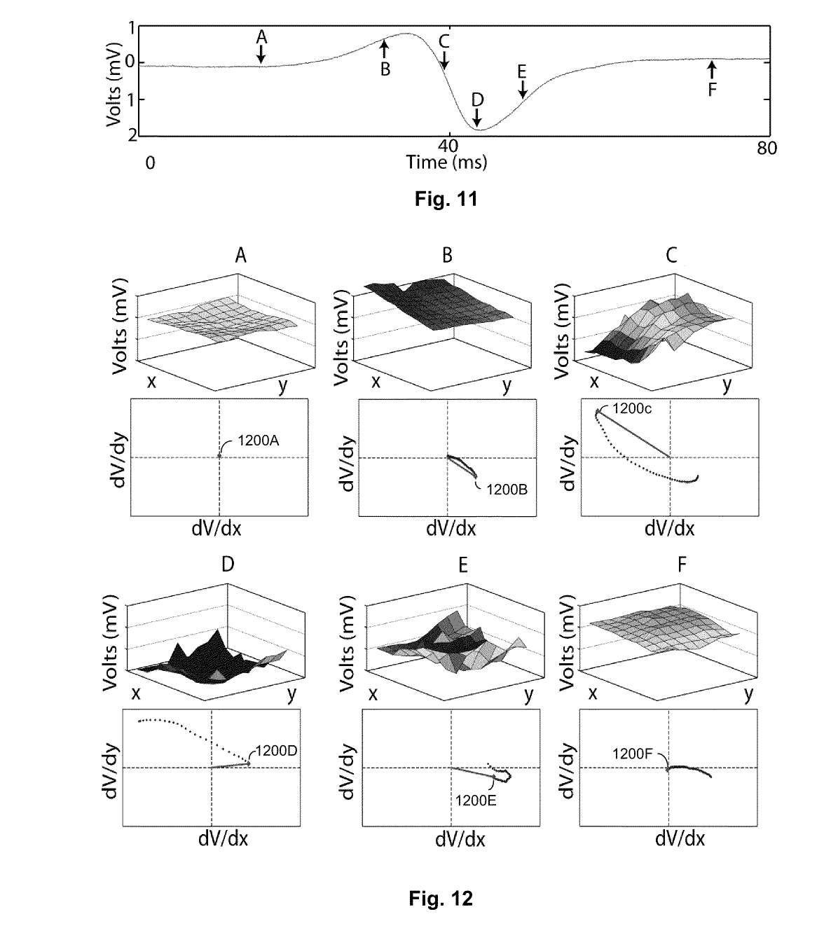

FIG. 11. shows an electrical trace of an interictal spike 1110 and multiple time points A-F on the interictal spike.

FIGS. 12A-12F show voltage mapped to the physical orientation of the interictal spike 1110 at the different time points A-E, respectively. Each of the FIGS. 12A-12F includes a three-dimensional plot (upper plot) showing the voltage level across the electrodes of the microelectrode array at the corresponding time point A-F, respectively, on the interictal spike. Each of the FIGS. 12A-12F includes a plot showing a voltage gradient across the plane of the array (x-y plane). The voltage gradient is a spatial derivative of voltage in two dimensions (x and y). In each plot, the point 1200A-1200F shows the current gradient at the corresponding time point A-E while the other points show previous gradients spaced by 300 μs is apart. This shows the propagation and directionality of the interictal spike moving across the array. FIG. 12C shows a maximal voltage gradient across the array at time point C, which may be used to determine the direction of propagation of the inert-ictal spike. For example, the direction of propagation may be based on a direction of the maximal voltage gradient.

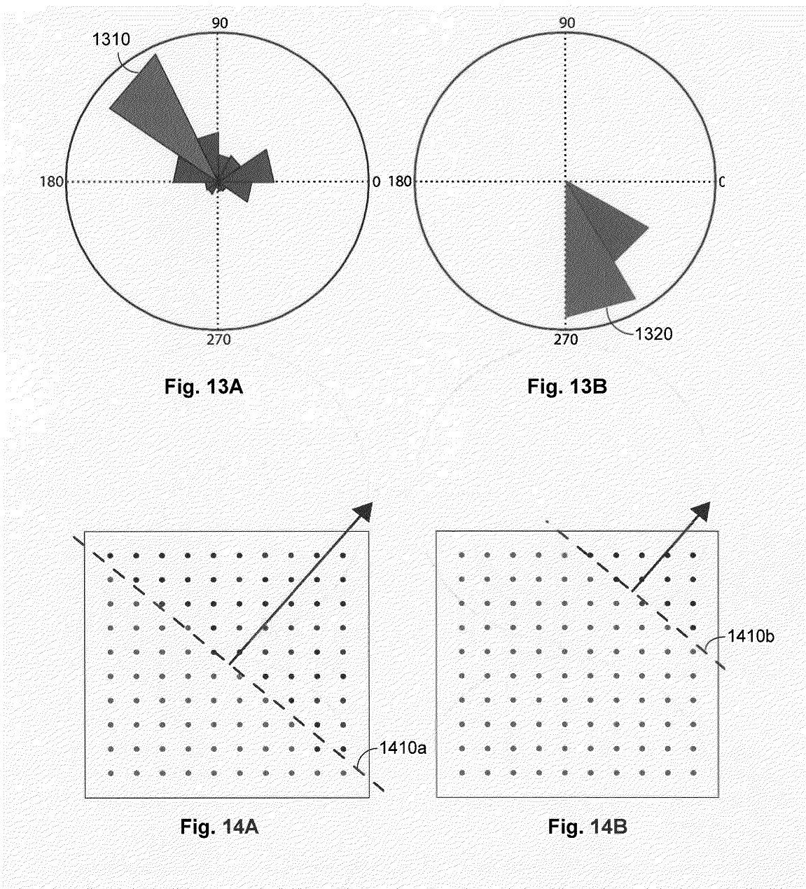

FIGS. 13A and 13B are histograms showing the directionality of multiple interictal spikes for two patients implanted with the microelectrode array. The direction for each interictal spike may be computed using the above technique. In the histograms, the computed directions for the interictal spikes are grouped into bins, where each bin corresponds to a range of directions (e.g., 30 degree range). The length of each bin indicates the number of measures directions falling within that bin. For example, in FIG. 13A, most of the computed directions fall within bin 1310, which corresponds to a range of 120 to 150 degrees. In FIG. 13B, most of the computed directions fall within bin 1320. The histograms in FIGS. 13A and 13B show that data recorded by the microelectrode array can be used to estimate the direction from which an epileptic activity is propagating.

FIGS. 14A and 14 illustrate a method for determining the direction of propagation of an interictal spike according to another embodiment of the invention. In this embodiment, the direction of propagation of interictal spikes and seizure activity is determined by analyzing the time of arrival of a wave front of an interictal spike across the microelectrode array. The arrival of the wave front at each electrode of the array may be determined by detecting when the electrical potential generated by the interictal spike at the electrode rises above a threshold voltage. As the interictal spike crosses the array, a boundary is formed between electrodes in which the threshold voltage has been crossed and electrodes in which the threshold has not been crossed. This boundary moves across the array as the interictal spike moves across the array. By determining the temporal progression of the boundary along a line orthogonal to the boundary, the direction of propagation of the interictal spike can be estimated by the direction along the line orthogonal to the boundary.

FIG. 14A shows a plot of the electrodes in the array at a certain time during the propagation of an interictal spike across the array. FIG. 14a shows a boundary 1410a between electrodes in which the electrical potential has exceeded the threshold voltage (left of boundary 1410a) and electrodes in which the electrical potential has not exceed the threshold voltage (right of boundary 1410a). The direction of propagation of the interictal spike may be determined by the direction orthogonal to the boundary 1410a.

FIG. 14B shows a plot of the electrodes at a later time during the propagation of an interictal spike across the array. FIG. 14b shows a boundary 1410b between electrodes in which the electrical potential has exceeded the threshold voltage (left of boundary 1410b) and electrodes in which the electrical potential has not exceed the threshold voltage (right of boundary 1410b). The direction of propagation of the interictal spike may be determined by the direction orthogonal to the boundary 1410b.

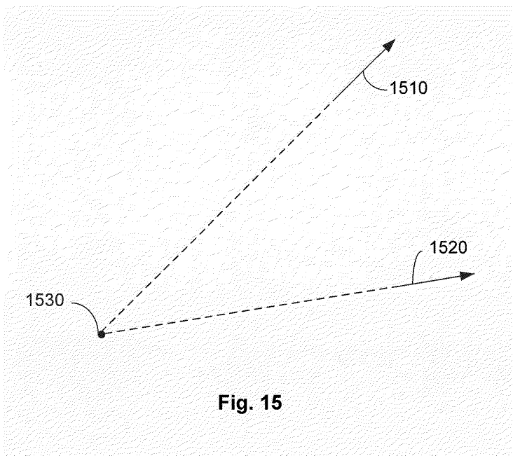

FIG. 15 illustrates a method for detecting the location of an epileptic focus from the direction of propagation of interictal spikes measured at two or more different locations of the cortex. FIG. 15 shows a direction 1510 of interictal spike propagation determined at a first location of the cortex. The direction 1510 may be a mean of directions of interictal spikes determined using a microelectrode array at the first location. The directions of the interictal spikes may be determined using any of the techniques discussed above or other techniques.

FIG. 15 also shows a direction 1520 of interictal spike propagation determined at a second location of the cortex. The direction 1520 may be a mean of directions of interictal spikes determined using a microelectrode array at the second location. The direction of interictal spike propagation may be determined at the two locations using two different microelectrode arrays that are implanted at the two locations and operate concurrently. Alternatively, the direction of interictal spike propagation may be determined at the two locations using the same microelectrode array, in which the array is positioned at the two locations at different times.

After the directions 1510 and 1520 of interictal spike propagation at the two locations are determined, the location 1530 of the epileptic focus generating the interictal spikes may be determined. This may be done by determining where two lines extending from the directions 1510 and 1520 at the two locations intersect, as shown in the example in FIG. 15. Although the direction of interictal spike propagation was determined at two locations in the example in FIG. 15 for ease of illustration, one skilled in the art will appreciate that the direction of interictal spike propagation may be determined at three or more different locations of the cortex. The location of the epileptic focus may be determined by triangulating the origin of interictal spikes based on the directions of the interictal spikes determined at the different locations. Other vector analyses may also be performed on the directions of the interictal spikes at the different locations to determine the location of the epileptic focus.

The location of the epileptic focus may allow a neurologist to target treatment from the epileptic pathology at the epileptic focus and/or position a microelectrode array or micro-ECog at or near the location of the epileptic focus to study the epileptic focus.

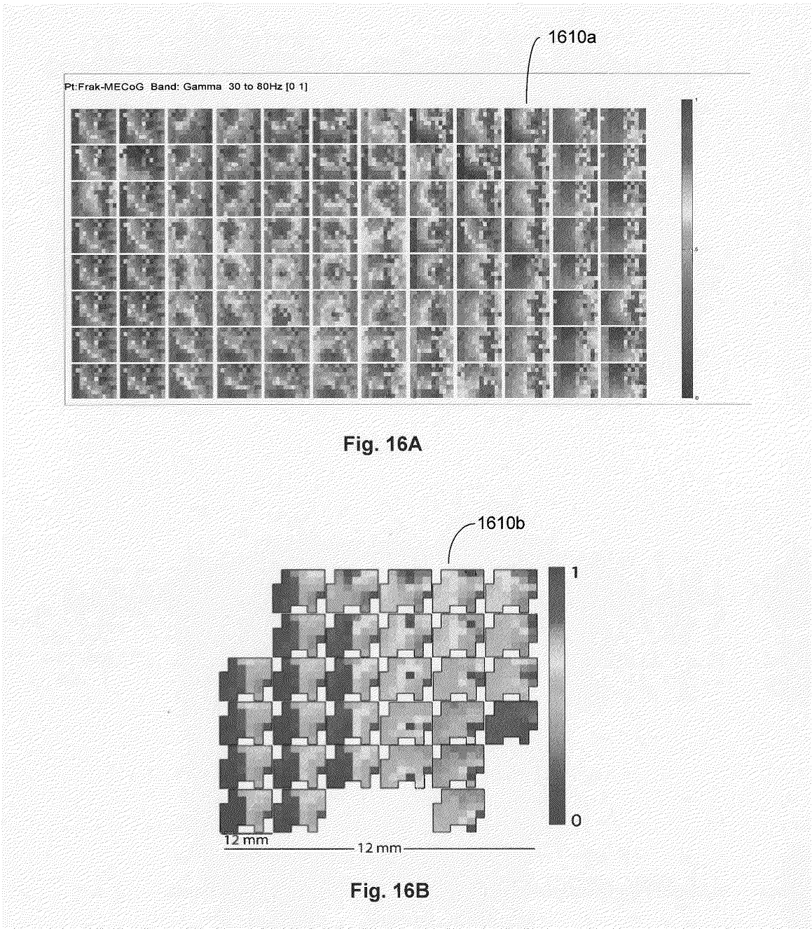

In one embodiment, correlation mapping between electrodes in an array may be used to map the boundaries between different areas of the cortex. FIGS. 16A and 16B show correlation analysis maps for an array implanted in the epidural space in a nonhuman primate and an array implanted in the subdural space in a human patient, respectively. In each correlation map, each square 1610a and 1610b show the correlation of the electrode at the corresponding location of the array with all other electrodes in the array. The square 1610a and 1610b at each location of the array comprises a miniature replica of the entire array indicating the pairwise cross-correlation of the electrode at that location to all other electrodes in the array. The location itself is identifiable in the miniature replica by the dark red pixel showing the autocorrelation (i.e., the electrode at the location correlated with itself).

Both of the arrays in FIGS. 16A and 16B where large enough that they spanned multiple cortical areas. Changes in the strength of correlations between the electrodes mapped onto the underlying cortical areas. In FIG. 16B, the array was implanted in the human patient over the primary motor and primary somatosensory cortexes. The left portion of the array was implanted over the primary motor cortex and the right portion of the array was implanted over the somatosensory cortex.

The correlation map in FIG. 16B shows a high correlation between electrodes in the left portion of the array corresponding to the primary motor cortex and lower correlation between electrodes in the right portion of the array corresponding to the samtosensory cortex. In FIG. 16B, the miniature replica at each electrode location in the left portion of the array shows high correlation between the electrode at that location and other electrodes located in the left portion of the array. Thus, the area of high correlation, indicated by the red on the left portion of the array, mapped onto the motor cortex, while the area of lower correlation on the right portion of the array mapped onto the somatosensory cortex.

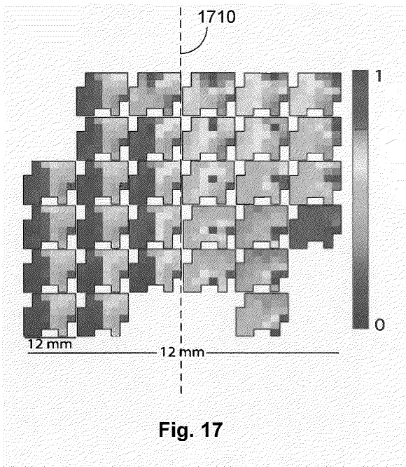

FIG. 17 shows a boundary 1710 between the area of high correlation and the area of lower correction, which corresponds to the boundary between the motor primary motor cortex and the somatosensory cortex. This demonstrates that the correlation mapping between electrodes in the array can be used to map boundaries between different cortical areas.

In one embodiment, the system in FIG. 1E or other system may perform correlation mapping to map different cortical areas of the brain. In this embodiment, the microelectrode area may be implanted over two cortical areas of the brain and the correlation mapping may correlate each electrode in the array with all other electrodes in the array. The system may then examine the resulting correlation map for areas of the array having different levels of correlation between electrodes. For example, the system may recognize two areas of the array in which the correlation between electrodes in one area of the array is significantly higher than the correlation between electrodes in the other area of the array. The system may then map a boundary between the two areas, which may correspond to a boundary between the two cortical areas of the brain. The microelectrode array may then be repositioned to map cortical areas in another part of the brain. This method can be used to map a boundary between healthy and pathological tissue in the brain.

The correlation mapping may also be used to map the boundaries of cortical areas that are used in various functions of the patient. For example, a microelectrode array may be implanted over an area of the brain and the patient may perform a certain function (e.g., motor movement, memory recall, etc.) while the microelectrode array takes readings from the area of the brain. The system may then perform correlation mapping, in which each electrode in the array is correlated with all other electrodes in the array. The system may then examine the resulting correlation map for an area indicating a high level of correlation between electrodes, which may correspond to a cortical area that is used to perform the function. The system may then identify the area with high correlation as an area of the brain that is used to perform the function. This can be used to determine neural activity in different areas of the brain correlated with specific functions performed by the patient.

It will be also appreciated by persons skilled in the art that numerous variations and/or modifications may be made to the specific embodiments disclosed herein, without departing from the scope or spirit of the disclosure as broadly described. The present embodiments are, therefore, to be considered in all respects illustrative and not restrictive of the present invention.

What is claimed is:

1. A method, of localizing a neurological pathology within the brain, comprising:
at each one of a plurality of electrode arrays located at or near the surface of a brain, detecting electrical activity occurring within the brain, wherein each of the electrode arrays comprises a plurality of electrodes;
for each one of the plurality of electrode arrays, determining a direction of propagation of the electrical activity based on the electrical activity detected by the electrode array; and
determining a location of the neurological pathology based on the determined directions of propagation of the electrical activity.

2. The method of claim 1, wherein the electrical activity comprises inter-itcal spikes and the neurological pathology comprises an epileptic focus.

3. The method of claim 1, wherein detecting the electrical activity occurring within the brain at each of the plurality of electrode arrays comprises detecting an electrical signal at each of the electrodes of the electrode array, and the determining the direction of propagation of the electrical activity at each one of the plurality of electrode arrays comprises:
determining a gradient of electrical signals across a plane of the electrode array based on the electrical signals detected by the electrodes of the electrode array; and
determining the direction of propagation of the electrical activity based on the determined gradient.

4. The method of claim 3, wherein the electrical signals comprise electrical potentials.

5. The method of claim 1, wherein detecting the electrical activity occurring within the brain at each of the plurality of electrode arrays comprises detecting an electrical signal at each one of the plurality of electrodes of the electrode array, and the determining the direction of propagation of the electrical activity at each one of the plurality of electrode arrays comprises:
determining a wave front of the electrical activity across the electrode array based on the electrical signals detected by the electrodes of the electrode array; and
determining the direction of propagation of the electrical activity based on the determined wave front.

6. The method of claim 5, wherein determining the wave front of the electrical activity across the electrode array comprises:
determining at which electrodes of the electrode array the respective electrical signal is above a threshold and at which electrodes of the electrode array the respective electrical signal is below the threshold;
forming a boundary between the electrodes corresponding to electrical signals above the threshold and the electrodes corresponding to electrical signals below the threshold; and
determining the wave front of the electrical activity based on the boundary.

7. A method, of characterizing neurological activity, comprising:
over a time period, detecting a local field potential occurring in a group of neurons;
during the time period, detecting action potentials within the group of neurons;
determining a value of a feature indicative of a relationship between the detected local field potential and the detected action potentials; and
outputting to an output device information indicative of the value of the feature.

8. The method of claim 7, wherein, if the value of the feature is different from a normal or standard range of values, outputting to the output device information indicative of the difference.

9. The method of claim 7, wherein the feature comprises a temporal correlation between the detected local field potential and the detected action potentials.

10. The method of claim 9, wherein the local field potential comprises an interictal spike.

11. The method of claim 9, wherein the value of the feature comprises one or more of an action potential firing rate and a number of action potentials during the detected local field potential.

12. The method of claim 7, wherein the feature comprises a temporal correlation between a phase of the detected local field potential and the detected action potentials.

13. The method of claim 7, wherein the feature comprises decrease in action potential firing rate during a time period following the local field potential.

14. The method of claim 13, wherein the local field potential comprises an interictal spike.

15. The method of claim 7, wherein the feature comprises a frequency of the local field potential.

16. The method of claim 15 wherein the feature comprises a temporal correlation between a phase of the detected local field potential and the detected action potentials.

17. The method of claim 8, further comprising outputting to the output device information indicative of a neurological state based on the difference.

18. The method of claim 8, further comprising outputting to the output device a differential diagnosis based on the difference.

19. A method, of mapping cortical areas in a brain using an electrode array comprising a plurality of electrodes, comprising:
at each electrode of the electrode array located at or near the surface of a brain, detecting an electrical signal from the brain;
correlating the electrical signals between different pairs of the electrodes of the electrode array to generate a correlation map of the electrode array; and
determining a boundary between a first cortical area and a second cortical area of the brain based on the correlation map.

20. The method of claim 19, wherein determining the boundary between the two cortical areas comprises:
identifying a first area of the correlation map indicating a higher level of correlation than a second area of the correlation map; and
mapping the first and second areas of the correlation map onto the first and second cortical areas of the brain, respectively.
